(19) United States
(12) Patent Application Publication
(10) Pub. No.: US 2018/0118640 A1
(43) Pub. Date: May 3, 2018

(54) METHOD FOR PURIFYING METHANE-COMPRISING GAS

(71) Applicant: Nederlandse Organisatie voor toegepast-natuurwetenschappelijk onderzoek TNO, 's-Gravenhage (NL)

(72) Inventors: EARL LAWRENCE VINCENT GOETHEER, 's-Gravenhage (NL); MARCO JOHANNES GERARDUS LINDERS, 's-Gravenhage (NL); LEONARDUS VOLKERT VAN DER HAM, 's-Gravenhage (NL)

(73) Assignee: Nederlandse Organisatie voor toegepast-natuurwetenschappelijk onderzoek TNO, 's-Gravenhage (NL)

(21) Appl. No.: 15/570,780

(22) PCT Filed: May 2, 2016
(86) PCT No.: PCT/NL2016/050312

§ 371 Date: Oct. 31, 2017

(30) Foreign Application Priority Data
May 1, 2015 (EP) 15166115.4

(51) Int. Cl. C07C 7/11; B01D 53/14; C10L 3/10

(52) U.S. Cl. CPC C07C 7/11; B01D 53/1425; B01D 53/1487; B01D 53/1493; C10L 2290/10; B01D 2252/20; C10L 2290/541; C10L 2290/12; C10L 3/101

(57) ABSTRACT

The invention is directed to the purification of gas streams comprising methane and hydrophobic pollutants. The invention provides a method for purifying a gas stream comprising methane and one or more hydrophobic pollutants comprising the steps of contacting the gas stream with a lean liquid stream comprising micelles of a surfactant or a separate surfactant phase. The resulting rich liquid stream comprises at least part of the hydrophobic pollutants. By subsequently changing the temperature of the rich liquid stream, a system of a pollutants-poor phase and a pollutants-rich phase is obtained. These phases are optionally separated and from the aqueous stream the lean liquid stream comprising micelles and/or surfactant can be regenerated.

The invention is in the field of gas purification, in particular purification of gas streams comprising methane and hydrophobic pollutants.

In particular the invention is related to a method wherein the gas stream is purified by contacting it with an aqueous liquid comprising a liquid and surfactants.

Gas streams comprising methane include for example biogas streams and natural gas streams. Such gas streams may contain hydrophobic pollutants which is undesirable for the use of these gas streams. Depending on the origin of the gas stream, the pollutants typically comprise siloxanes, organo sulfur components, hydrocarbons comprising more than five carbon atoms, such as terpenes and aromatics (e.g. BTX, i.e. benzene, toluene and xylene) in an amount that is typically in the range of 0-10000 ppm. Such pollutants are undesirable for several reasons. For instance, the presence of siloxanes may result in the formation of silicon dioxide (silica) when the gas stream is combusted. Silicon dioxide is a powder that may be deposited on equipment and cause a decrease in efficiency and an increase in maintenance cost. Terpenes and aromatics may for instance lead to deterioration of polymeric materials which are e.g. present in seals in the gas grid pipelines and/or because of their odor mask the odor of functional odorants, such as tetrahydrothiophene and tert-butylthiol that are normally added to the gas grid for safety. Hence, prior to the use of methane comprising gas streams, the gas streams should normally first be purified by removing most of these pollutants. Current purification methods typically rely on adsorbents, such as activated carbon or alumina. However, such adsorbents generally do not adsorb all pollutants with the desired effectiveness and are expensive as they are typically discarded after use. Hence, these methods are both economically and operationally undesired.

It is therefore desired to obtain an improved method for purifying a methane comprising gas stream that contains hydrophobic pollutants.

It was found that this object can be met by a method that comprises the subsequent steps of:

a) contacting said gas stream with a lean liquid comprising an aqueous liquid and a surfactant to obtain a purified gas stream and a rich liquid comprising at least part of said hydrophobic pollutants, wherein the temperature of the lean liquid is above the critical micelle temperature;

b) changing the temperature of the rich liquid to obtain a pollutants-poor phase and a pollutants-rich phase;

c) optionally separating said pollutants-poor and pollutants-rich phases to obtain a separated aqueous stream comprising said liquid and a separated pollutants stream comprising said pollutants; and

d) regenerating said lean liquid comprising surfactants.

Step c) is optional, in that it is not necessary to separate the two phases first. It is thus also possible to remove the pollutants by stripping them from the pollutants-rich phase while this phase is still in the presence of the pollutants-poor phase, as is the case in step b).

The invention is based on the ability of the surfactant to form a separate surfactant phase or micelles in the aqueous liquid. These micelles or surfactant phase provide a hydrophobic environment in the lean liquid so it may absorb the hydrophobic pollutants. In this respect, with hydrophobic pollutants is meant pollutants that have a logP-value of at least 0.5. The logP-value expresses the partition coefficient that is the logarithm of the ratio of concentrations (on a weight basis) of the pollutants when allowed to separate between octanol and water.

The invention is thus directed to the purification of gas streams comprising methane and hydrophobic pollutants. The invention provides a method for purifying a gas stream comprising methane and one or more hydrophobic pollutants comprising the steps of contacting the gas stream with a lean liquid comprising micelles of a surfactant or a surfactant phase. The resulting rich liquid comprises at least part of the hydrophobic pollutants. By subsequently changing the temperature of the rich liquid, a pollutants-poor phase and a pollutants-rich phase are obtained. These phases can be separated and from the aqueous stream the lean liquid comprising micelles and/or surfactant can be regenerated.

The micelles in the lean liquid can be obtained by carrying out step a) at a temperature above the critical micelle temperature. As such, micelles are formed. If the temperature is increased further, i.e. above the cloud point temperature, a separate surfactant phase is formed. Both the micelles and the separate surfactant phase form the above-described hydrophobic environment in which the hydrophobic pollutants may absorb. In the case the separate surfactant phase is formed and used, it is highly beneficial that there is a large surface area between the surfactant-depleted and surfactant phase, in order to ensure fast transfer rates. This can typically be achieved by agitation (e.g. stirring) of the liquid and/or selecting the appropriate temperature and/or the relative density of the aqueous liquid and the surfactants. If the densities of the aqueous liquid and the surfactants are about equal, a turbid or clouded system is typically obtained with a large surface area.

By contacting the polluted gas stream with a lean liquid comprising an aqueous liquid and the surfactant, the pollutants are absorbed by the liquid and the purified gas stream can be obtained. Furthermore, a rich liquid comprising at least part of said hydrophobic pollutants is obtained.

Advantageously, the invention includes the regeneration of the lean liquid from the rich liquid by utilizing the temperature sensitivity of the surfactant, and concomitantly the temperature sensitivity of the micelles or the surfactant phase. The surfactant in accordance with the present invention is temperature sensitive which means that by changing the temperature, the phase of the surfactant in the aqueous liquid may change. For instance, at a low temperature the surfactant may be freely solvated by the liquid (i.e. a solution of the surfactant is formed). Upon increasing the temperature of these freely solvated surfactants and passing the critical micelle temperature, the surfactant may form micelles, which is a different phase than the freely solvated surfactant. Further increasing the temperature and passing the cloud point temperature may cause the surfactant to form a liquid phase that is a separate phase from the liquid in which the surfactant was previously formed. Hence, two phases are formed: a surfactant-lean phase and a surfactant-rich phase. The surfactant rich phase is herein also simply referred to as surfactant phase. Each dissolved surfactant typically has its own phase diagram from which the critical micelle and cloud point temperatures may be determined. It will be appreciated that all phase transitions are reversible. The phase diagram of the water-surfactant mixtures also includes freezing and boiling temperatures. In some cases, the boiling temperature is lower than the cloud point temperature, which in practice means that there is no cloud point temperature. Similarly, the critical micelle temperature may be absent, in the case that the freezing temperature is higher than the critical micelle temperature would be. All phase change temperatures are also dependent on the concentration and type of the surfactant(s) and other additives. The method according to the present invention can be adjusted depending on the phase diagram of the liquid system that is used.

In the case that a liquid system is used that does not have a critical cloud point temperature, the temperature change in step c) may comprise lowering the temperature to below the critical micelle temperature.

In the case that a liquid system is used that does not have a critical micelle temperature, the temperature change in step c) may comprise increasing the temperature above the critical cloud point temperature.

The inventors found that by changing the temperature of the rich liquid, a pollutants-poor phase and a pollutants-rich phase are obtained. It may depend on whether the temperature of the rich liquid is increased or decreased which types of phases are obtained. Additionally, in the case that the concentration of pollutants in the gas stream is low, the formation of a pollutants-rich phase may not be visible to the naked eye.

For instance, by decreasing the temperature of the liquid below the critical micelle temperature, the surfactant changes from the micellar phase to the dissolved phase, or in the case that step a) was carried out at a temperature above the critical cloud point temperature, the surfactant phase changes to the micellar phase, and then to the dissolved phase. Consequently, the hydrophobic environment previously provided by the micelles or the surfactant phase is substantially lost. As a consequence, a pollutants-rich phase and a pollutants-poor phase comprising the surfactant and aqueous liquid are obtained. Hence, the majority of the surfactant is typically in another phase than the majority of the pollutants. Typically, in a sequential step (step c), said pollutants-poor and pollutants-rich phases are separated to obtain a separated aqueous stream comprising the liquid and the surfactant and a separated pollutants stream comprising said pollutants. Alternatively, step b) and step c) can be carried out simultaneously, e.g. the temperature is decreased while the phases are separated (i.e. by providing a gas flow through the liquid). It may also be the case that steps b), c) and d) are carried out simultaneously, e.g. decreasing the temperature is carried out during phase separation while one phase (typically the pollutants-rich phase) evaporates such that the lean liquid is regenerated.

In the case that step a) was carried out at a temperature above the critical cloud point temperature, it is preferred that the temperature in step b) is changed to below the critical micelle temperature.

Upon reheating the separated aqueous stream comprising the liquid and the surfactant above the critical micelle temperature and possible further to above the cloud point temperature, micelles and/or possibly a surfactant phase are formed and the lean solution is regenerated.

When the temperature of the rich liquid is increased above the cloud point temperature, the liquid becomes clouded as the surfactant forms a separate phase from the aqueous liquid phase. Hence, a surfactant-rich phase and the aqueous liquid phase are formed. It is noted that although the surfactant-rich phase is formed, part of the surfactant may still be present in the aqueous liquid phase. The surfactant-rich phase is typically more hydrophobic than the surfactant-poor phase and thus the surfactant-rich phase is typically the pollutants-rich phase, while the aqueous liquid phase is typically the pollutants-poor phase. However, depending on the hydrophilicity of the surfactant, this may also be reversed.

Increasing the temperature in step b) is typically carried out if the temperature in step a) is below the cloud point temperature, such that phase change is obtained. However, it may also be possible to increase the temperature further above the cloud point temperature since this results in further separation of the surfactant and the aqueous liquid (i.e. the relative amount of surfactant phase increases), which can be used in subsequent steps.

In a sequential step (step c), said pollutants-poor and pollutants-rich phases are separated to obtain a separated aqueous stream comprising the liquid and the surfactant and a separated pollutants stream comprising said pollutants. In case the pollutants-rich phase comprises large amounts of surfactant, the separated pollutants stream also comprises large amounts of surfactant. The pollutants are preferably separated from these components by conventional techniques such as evaporation or precipitation to recover the surfactant. The recovered surfactant may advantageously be recycled by e.g. rejoining the separated aqueous stream. Cooling the separated aqueous stream below the cloud point temperature typically results in micelles formation thus regeneration of said lean solution.

As described herein above, by changing the temperature of the rich liquid a system comprising the pollutants-poor phase and the pollutants-rich phase is obtained. In order to perform the step of separating these phases into the separated aqueous stream comprising said liquid and the separated pollutants stream comprising said pollutants, the pollutants-poor and pollutants-rich phases are typically allowed to equilibrate and split into layers by using their intrinsic difference in density. The addition of additives such as inorganic salts or co-liquids may influence the density of at least one of the phases to facilitate the splitting into layers. The splitting may also be facilitated by centrifugation.

The step of providing micelles and/or a separate surfactant phase in the separated aqueous stream to regenerate said lean liquid comprising micelles, typically comprises changing the temperature of the separated aqueous stream in which the surfactant is present. This surfactant may be the recovered surfactant or may be the surfactant that was present in the pollutant-poor phase or combinations thereof.

The pollutants typically comprise one or more compounds selected from the group consisting of siloxanes, organo sulfur components, volatile hydrocarbons comprising more than five carbon atoms (such as terpene and/or aromatics) and combination thereof. Typical aromatics are benzene, toluene and xylene. However, other hydrophobic compound may also be removed. The absorbance capacity of the lean liquid typically depend on i.a. the concentration of surfactant, temperature and pressure of the gas stream. Also the type of surfactant may influence the absorbance capacity.

The micelles of the present invention may be formed by one type of surfactant or by combinations of different types of surfactant as described herein. The surfactant in accordance with the present invention may be an ionic surfactant (e.g. sodium lauryl sulfate) or a non-ionic surfactant. Surfactants in aqueous systems are known to self-assemble into larger assemblies called micelles. These micelles may be shaped in all different kinds of shapes such a sphere, ellipsoid, cylinder, unilamellar vesicle, planar structure and the like. The common feature is the formation of a hydrophobic environment in the aqueous system. In the context of the present invention, the term micelle covers all shapes of surfactant assemblies by which a hydrophobic environment is formed.

Several non-ionic surfactants are known in the art. Preferably, the non-ionic surfactants are selected from the group consisting of polyoxyethylene glycol alkyl ethers (e.g. Brij®), polyoxypropylene glycol alkyl ethers, glucoside alkyl ethers, polyoxyethylene glycol octylphenol ethers (e.g. Triton® X-100), polyoxyethylene glycol alkylphenol ethers (e.g. Nonoxynol-9), glycerol alkyl esters (e.g. glyceryl laurate), polyoxyethylene glycol sorbitan alkyl esters, sorbitan alkyl esters, cocamide monoethanolamine (MEA), cocamide diethanolamine (DEA), dodecyldimethylamine oxide, block copolymers such as polyethylene glycol and polypropylene glycol, polyethoxylated tallow amine and combinations thereof.

Typical ionic surfactants that may be used in accordance with the present invention may be anionic and selected from the group consisting of alkylated sulfates, sulfonates, phosphates, carboxylates and combinations thereof.

It was found that certain block copolymers are particularly suitable for the present invention. These block copolymers comprise hydrophobic and hydrophilic blocks which result in self-assemblies in aqueous systems.

A particularly preferred type of non-ionic block copolymeric surfactant is one or more types of poloxamers. These poloxamer are triblock copolymers wherein the middle block is hydrophobic poly(propyleneoxide) (PPO) and the two outer blocks are hydrophilic poly(ethyleneoxide) (PEO). Poloxamers are commercially available under trade names such as Synperonic®, Pluronic® and Kolliphor®. By variation of the poloxamer chain length and the ratio of the mass of the hydrophobic and hydrophilic block, the physical properties of the poloxamer may be adjusted.

Poloxamers are commonly named using a code consisting of a letter followed by two or three numbers. For Pluronics®, the letter represents the state of the pure Pluronic® at ambient conditions; either liquid (L), wax/paste (P), or solid/flakes (F). The last number multiplied by 10 gives the weight percentage of PEO, and the remaining one or two numbers multiplied with 300 g/mol represent the molar weight of the PPO group. For example, L31 is a liquid component with a PPO molar weight of about 900 g/mol containing 10 wt % PEO, while F108 is a solid component with a PPO molar weight of about 3000 g/mol containing 80 wt % PEO. For sake of clarity and conciseness, the nomenclature of Pluronics® will be used herein, also when referring to other poloxamers.

Typically, dissolved surfactants tend to foam when being agitated by e.g. stirring or transportation. In certain embodiments foaming can be used to enhance mass transfer of hydrophobic components towards the liquid phase. However, in a typical embodiment foaming is less desired and thus preferably minimized. The foaming behavior of the surfactants can be modified by their chemical structure. The amount of foaming can be determined by the Ross-Miles method (DIN 53902 Part 2, or ASTM D 1175-53) using 0.1 wt % solution at 50° C. Also the cloud point and critical micelle temperatures typically depend on the chemical structure of the surfactant. The cloud point temperature of the liquid comprising micelles can routinely be determined by slowly heating the liquid when it is clear until the liquid visibly becomes clouded. Alternatively, the cloud point temperature can be determined by slowly cooling a clouded liquid until the liquid becomes clear. The critical micelle temperature of the liquid comprising micelles can routinely be determined by cooling the clear liquid and using light scattering and/or fluorescence spectroscopy to monitor the presence of micelles (see e.g. Alexandridis et al., Colloids and Surfaces A: Physicochemical and Engineering Aspects 96(1995) 1-46).

Preferably, the weight percentage of PEO in the poloxamer is less than 50%, more preferably less than 30%, most preferably between 5% and 25%. It was found that these preferred poloxamers are particular suitable for the present invention for their low tendency to foam, favorable cloud point and critical micelle temperature, viscosity and absorbance capacity. Examples of preferred poloxamers are L62, L92, L31, L61 and L81, and combinations thereof.

The cloud point and/or critical micelle temperature may also be influenced by the addition of additives. These additives can be added to the lean liquid before or after step a).

The additive may be inorganic salts, hydrotropes, anionic and cationic surfactants, organic liquids and the like. Inorganic salts that may be added are for instance Na₂SO₄, NaF, NaCl, NaBr, NaI, and/or NaSCN. Hydrotopes that may be added are for instance sodium benzene sulfonate, sodium toluene sulfonate, sodium xylene sulfonate, sodium p-chloro-benzene sulfonate, sodium taurate and/or sodium sulfanilate. Alkanoates that may be added are sodium caprate, sodium caprylate, sodium caproate, sodium valerate, sodium butyrate, sodium 6-amino caproate, sodium acetate, and/or sodium 4-amino butyrate. Carboxylates that may be added are sodium oxalate, sodium benzoate, sodium salicylate, sodium succinate and/or sodium phthalate.

The addition of an anionic and/or a cationic surfactant to the liquid comprising micelles of non-ionic surfactant typically increases the cloud point temperature. Anionic surfactant that may be used as additives are sodium dodecyl sulfate, sodium decyl sulfate, sodium decane sulfonate, sodium dodecyl benzene sulfonate and sodium dodecyl sulfate. Cationix surfactant that may be used as additives are alkyltrimethylammonium bromides (TABr) such as C₁₀TABr, C₁₂TABr, C₁₄TABr, C₁₆TABr, C₁₈TABr and C₁₂TABr.

Organic liquids that may be used as additives are alcohols such as methanol, ethanol, 1-propanol, 1-butanol, 2-butanol, 1-pentanol, 2-methoxyethanol, 2-ethoxyethanol, 2-butoxyethanol and other water-miscible liquids such as methyl acetate and ethyl acetate.

Step a) of contacting said gas stream with a lean liquid preferably takes place at a temperature and pressure close to the temperature of the gas stream. This i.a. reduces the energy and time requirements of the procedure. In case the gas stream is a biogas stream, this is typically about 20 to 40° C. and at about atmospheric pressure. Hence, in a preferred embodiment, step a) takes place at a temperature between 0 to 50° C. and/or at a pressure of about atmospheric pressure.

The cloud point and/or critical micelle temperature are preferably close to the temperature at which step a) takes place. This limits the required temperature change in step b). Therefore, changing the temperature in step b) preferably means increasing or decreasing the temperature with maximum 50° C., more preferably with maximum 20° C., most preferably with maximum 10° C.

The temperature at which step a) takes place may be closer to the cloud point temperature than to the critical micelle temperature. Therefore, the required temperature change when heating is typically smaller than when cooling. A lower temperature change is beneficial for energy and time consumption. Hence, in a preferred embodiment changing the temperature in step b) is increasing the temperature of the rich aqueous liquid.

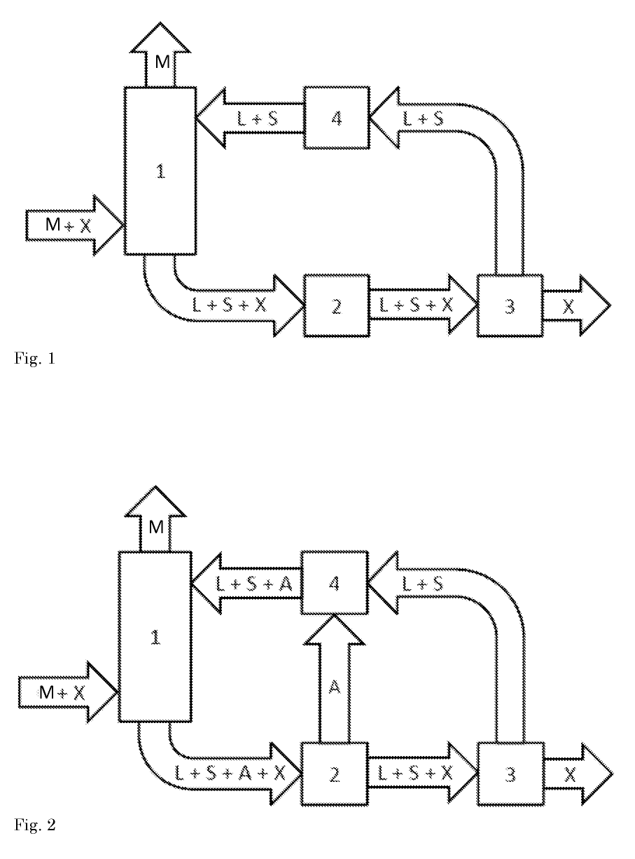

In a particular embodiment of the present invention, as schematically illustrated by FIG. 1, the gas stream comprising methane (M) and hydrophobic pollutants (X) is contacted with the lean liquid comprising the aqueous liquid (L) and surfactant (S) in a contacting zone 1. A purified gas stream comprising methane (M) and a rich liquid comprising at least part of said hydrophobic pollutants (L+S+X) are produced. The rich liquid is cooled below the critical micelle concentration in zone 2 to obtain a system comprising a pollutants-poor phase and a pollutants-rich phase. In zone 3 is the pollutants-rich phase separated from the pollutants-poor phase and a separated pollutants stream (X) and a separated aqueous stream comprising the liquid and surfactant (L+S) are obtained. The separated aqueous stream is re-heated in zone 4 to provide micelles and/or a surfactant phase and the lean liquid is regenerated. Heat integration between zones 2 and 4 can be used to increase the energy efficiency of the process.

FIG. 2 illustrates schematically a specific variation to the embodiment that is illustrated by FIG. 1. In this specific variation, an additive (A) is present in the lean liquid when this stream is contacted with the gas stream comprising methane (M) and hydrophobic pollutants (X). During cooling in zone 2, this additive influences the critical micelle concentration of the rich liquid. During the cooling in zone 2, the additive may precipitate and be separated so it may be recycled (e.g. by addition during re-heating of the separated aqueous stream in zone 4).

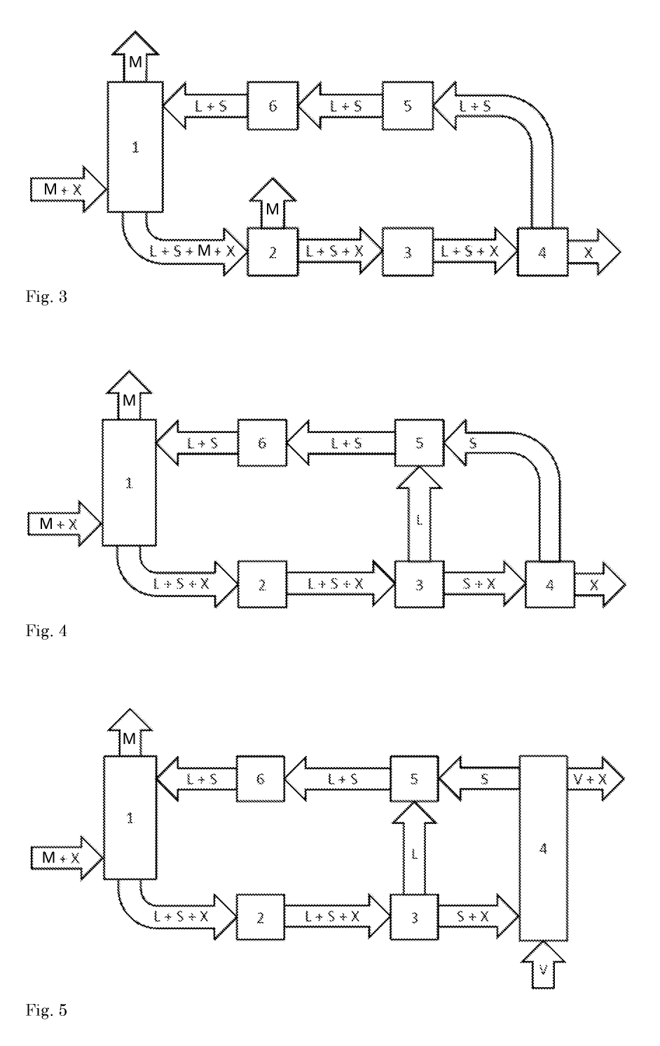

In a further particular embodiment of the present invention, as schematically illustrated by FIG. 3, after contacting the lean liquid with the gas stream, some methane may remain trapped in the rich liquid stream. This methane is preferably recovered by e.g. flashing, heating and/or using a stripping gas in zone 2.

In another particular embodiment of the present invention, the rich liquid may be heated. This embodiment is schematically illustrated by FIG. 4. This embodiment is a variation on the embodiment that is illustrated by FIG. 1 and differs in that in zone 2 the rich liquid is heated above the cloud point temperature instead of cooled below the critical micelle concentration. This typically results in a pollutants-poor phase comprising the aqueous liquid and a pollutants-rich phase comprising the majority of the hydrophobic pollutants and large amounts of the surfactant. After separation of both phases in zone 3, a separated pollutants stream and an aqueous liquid are obtained. The separated pollutants stream is subsequently separated in zone 4 into a surfactant stream and a pollutant stream, for example by heating, to evaporate the hydrophobic pollutants to recover the surfactant. The recovered surfactant is then typically mixed with said aqueous liquid in zone 5 such that after cooling in zone 6 the lean liquid is recovered. The energy efficiency of the process can be increased by heat integration between the cooling in zone 6 and the heating in zone 2 and/or zone 4.

In a further embodiment (not shown) an additive is added before the rich liquid is heated in zone 2 to influence the cloud point temperature. The additive is typically water soluble and therefore remains with the aqueous liquid after separating the pollutants-rich and pollutants-poor phases. After recombination of the recovered surfactant with said aqueous liquid and during cooling to regenerated the lean liquid in zone 6, the additive may precipitate, be recovered and recycled.

In a particular embodiment wherein the pollutants-rich phase comprises the majority of the hydrophobic pollutants and large amounts of the surfactant, instead of heating the separated pollutants stream to recover the surfactant, surfactant may be recovered by stripping the separated pollutants stream with a stripping stream (V) in zone 4, as illustrated by FIG. 5.

For the purpose of clarity and a conciseness, features are described herein as part of the same or separate embodiments. However, it will be appreciated that the scope of the invention may include embodiments having combinations of all or some of the features described. For instance, embodiments comprising the cooling step below the micelle concentration or heating above the cloud point temperature in combination with utilizing the additive, gas recovery, heat integration/recovery and/or stripping are all included and described herein.

The invention may further be illustrated by the following Examples.

### EXAMPLE 1

200 g aqueous solution of 1 wt % Pluronic L31 was prepared having a cloud point temperature of about 40° C. The solution was heated to 35° C. and a gas mixture, which contained about 2 g/m³ pinene (pollutant) in a 100 ml/min N₂ flow, was continuously added to the solution. The gas was dispersed into the liquid as small bubbles using a sparger. The contact time between the gas and the liquid was estimated to be less than 1 second. At given time intervals, the solution was visually inspected to assess the presence of a foam layer, and the pinene concentration in the outlet gas was measured using gas-chromatography. Almost complete pinene removal was observed at the start of the experiment, and negligible foaming was observed during the entire experiment. As time progressed, the outlet pinene concentration increased, reaching a more or less stable concentration after 49 min.

### EXAMPLE 2

Example 1 was repeated, but as aqueous solution a 1 wt % Pluronic L81 having a cloud point of 19° C. was used. The solution was at room temperature while the gas mixture containing about 2 g/m³ of pinene in a N2 flow of 100 ml/min was continuously added to the solution. Results were comparable to Example 1 and summarized in Table 1.

The results from Examples 1 and 2 illustrate the absorbance ability of the surfactant solutions to remove terpenes contaminants from a gas stream.

TABLE 1

| Example | 1 | 2 |
|---|---|---|
| Surfactant type | Pluronic L31 | Pluronic L81 |
| Surfactant concentration (wt %) | 1 | 1 |
| Solution cloud point temperature (° C.) | 40 | 19 |
| Solution initial temperature (° C.) | 35 | 20 |
| Solution mass (g) | 200 | 200 |
| Gas flow (ml/min) | 100 | 100 |
| Contaminant type | Pinene | Pinene |
| Contaminant feed concentration (g/m³) | 1.7 | 1.8 |
| Minimum contaminant outlet concentration | <0.05 | <0.05 |
| (g/m³) | | |
| Stabilization time for contaminant outlet | 49 | 34 |
| concentration (min) | | |

### EXAMPLE 3

Three liter of aqueous solution of 5 wt % Pluronic L81 was prepared having a cloud point temperature of about 17° C. The solution was heated to 25° C. obtaining a clouded system comprising a dispersion of an aqueous solution and a surfactant-rich phase. A gas mixture, which contained about 0.8 g/m3 limonene (pollutant) in a 500 ml/min N₂ flow, was continuously added to the solution. The gas was dispersed into the liquid as small bubbles using a sparger. The contact time between the gas and the liquid was estimated to be less than 10 seconds.

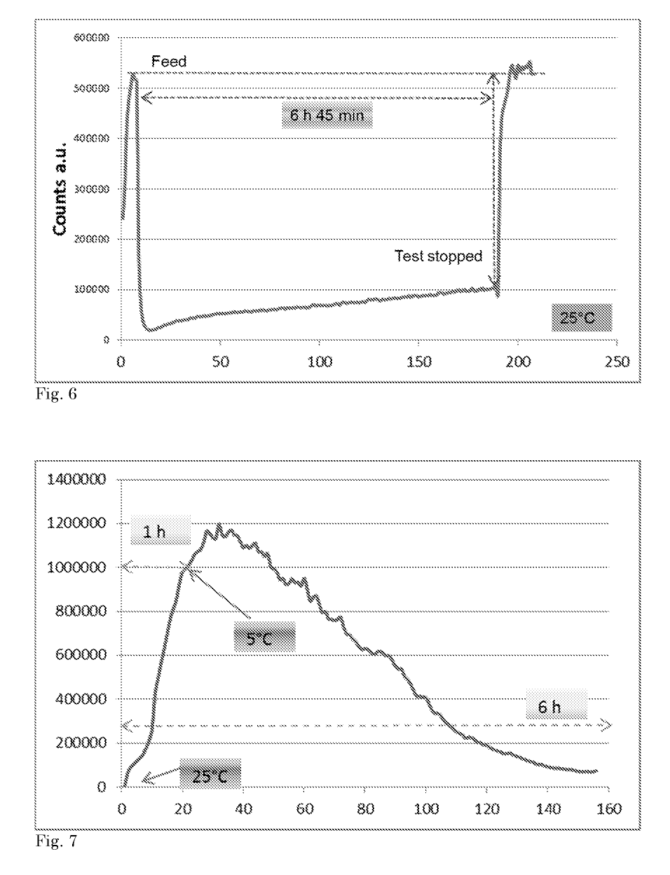

At given time intervals, the solution was visually inspected to assess the presence of a foam layer, and the limonene concentration in the outlet gas was measured using gas-chromatography (see FIG. 6). Almost complete limonene removal was observed at the start of the experiment, and negligible foaming was observed during the entire experiment. As time progressed, the outlet limonene concentration slowly increased, reaching a level of about 20% of the feed concentration after 6 hours 45 min, upon which the absorption experiment was stopped. It is noted that the conditions in this test are such that the solution absorbs the pollutant above the cloud point temperature.

Subsequently, the solution was cooled to 5° C., obtaining a clear solution, and a 500 ml/min N2 flow was continuously added to the solution, acting as a purge flow. As detected by gas-chromatography (see FIG. 7), this caused the release of limonene from the solution, at even higher concentrations than the feed concentration during absorption, indicating a fast release of the contaminant limonene. After 6 hours of purging, the limonene concentration in the outlet gas flow was almost zero again, indicating that the absorption solution was regenerated and suitable for absorption again.

### EXAMPLE 4

In order to determine the cloud point temperatures of various surfactant solutions, and the effect that additives can have on this temperature, cloud point measurements have been performed. For about 1 mL of solution of known composition, the temperature was slowly (0.25° C./min) increased and decreased within a predefined temperature range, while continuously stirring and monitoring the solution's light transmission properties. The cloud point temperature could be detected by a sudden change in light transmission. Table 2 gives an overview of the performed cloud point measurement results.

TABLE 2

| Surfactant | | Additive | | Cloud point |
|---|---|---|---|---|
| Type | Concentration | Type | Concentration | Temperature |
| L31 | 5 wt % | — | — | 32.7° C. |
| L31 | 20 wt % | — | — | 25.0° C. |
| L81 | 1 wt % | — | — | 20.6° C. |
| L81 | 5 wt % | — | — | 19.4° C. |
| L81 | 10 wt % | — | — | 17.1° C. |
| L81 | 5 wt % | SDS | 29 mg/kg | 22.1° C. |
| L81 | 5 wt % | SDS | 91 mg/kg | 32.9° C. |
| L81 | 10 wt % | SDS | 29 mg/kg | 26.9° C. |
| L81 | 10 wt % | SDS | 91 mg/kg | 31.3° C. |
| L81 | 10 wt % | SDS | 288 mg/kg | 36.3° C. |

The results indicate that the cloud point temperature of the solutions can i.a. be increased by: 1) decreasing the size of the Pluronic molecules, 2) decreasing the concentration of the surfactant, 3) increasing the concentration of the additive SDS (sodium dodecyl sulfate).

1. A method for purifying a gas stream comprising methane and one or more hydrophobic pollutants having a logP-value of at least 0.5, said method comprising the steps of
a) contacting said gas stream with a lean liquid stream comprising an aqueous liquid and a surfactant to obtain a purified gas stream and a rich liquid stream comprising at least part of said hydrophobic pollutants, wherein the temperature of the lean liquid is above the critical micelle temperature;
b) changing the temperature of the rich liquid stream to obtain a pollutants-poor phase and a pollutants-rich phase;
c) optionally separating said pollutants-poor and pollutants-rich phases to obtain a separated aqueous stream comprising said liquid and a separated pollutants stream comprising said pollutants; and
d) regenerating said lean liquid comprising surfactants.

2. The method according to claim 1 wherein the hydrophobic pollutants comprise one or more compounds selected from the group consisting of siloxanes, organo sulfur components, hydrocarbons comprising more than five carbon atoms and combinations thereof.

3. The method according to claim 1 wherein the surfactant is a non-ionic surfactant.

4. The method according to claim 1 wherein changing the temperature in step b) is increasing the temperature of the rich aqueous liquid to above the cloud point temperature of the liquid.

5. The method according to claim 1, wherein changing the temperature in step b) is decreasing the temperature to below the critical micelle temperature of the liquid.

6. The method according to claim 1 wherein said lean aqueous liquid further comprises an additive that influences the cloud point temperature and/or critical micelle temperature.

7. The method according to claim 1 wherein step b) further comprises the addition of an additive that influences the cloud point temperature and/or the critical micelle temperature of the rich liquid.

8. The method according to claim 1 wherein step a) takes place at a temperature between 0 to 50° C. and/or at a pressure of about atmospheric pressure.

9. The method according to claim 1 wherein the step of regenerating said lean liquid comprises changing the temperature of the separated aqueous stream in which the surfactant is present.

10. The method according to claim 1 wherein the surfactant is a block-copolymer.

11. The method according to claim 1 wherein the surfactant is a poloxamer.
